(19) United States
(12) Patent Application Publication
(10) Pub. No.: US 2005/0239816 A1
(43) Pub. Date: Oct. 27, 2005

(54) Amonafide salts

(75) Inventors: Alfred M. Ajami, Brookline, MA (US); David O. Barlow, Byfield, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Xanthus Life Sciences, Inc., Cambridge, MA (US)

(21) Appl. No.: 11/170,515

(22) Filed: Jun. 29, 2005

Related U.S. Application Data
(63) Continuation-in-part of application No. 10/690,458, filed on Oct. 20, 2003.
Application No. 10/690,458 is a continuation-in-part of application No. PCT/US03/12619, filed on Apr. 22, 2003.
Application No. PCT/US03/12619 is a continuation-in-part of application No. 10/128,129, filed on Apr. 22, 2002, now Pat. No. 6,693,198.

(51) Int. Cl. C07D 471/06; A61K 31/473

(52) U.S. Cl. 514/296; 546/98

(57) ABSTRACT

Disclosed are organic hydroxy acid salts of amonafide: 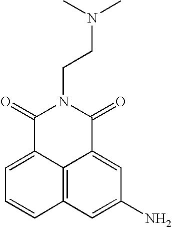 Also disclosed are methods of preparing salts of amonafide and method of treating subjects suffering from cancer.

### RELATED APPLICATION

This application is a continuation-in-part of U.S. patent application Ser. No. 10/690,458, filed Oct. 20, 2003, which is a continuation-in-part of International Application No. PCT/US03/12619, which designated the United States and was filed Apr. 22, 2003, published in English, which is a continuation-in-part of and claims priority to U.S. patent application Ser. No. 10/128,129, filed Apr. 22, 2002. The entire teachings of the above applications are incorporated herein by reference.

### BACKGROUND OF THE INVENTION

Traditional pharmaceutical process technology for manipulating the physical properties and water solubility of these amonafide and related compounds has been to render them water soluble with strong mineral acids. In this process, salt formation is reserved as the final step in the synthesis, as described by Brana and associates (U.S. Pat. No. 5,420,137; 1995). Such mono and divalent mineral acid salts retain hygroscopicity, and the divalent species, which form hydrates, have also been found to be incompatible with many pharmaceutical auxiliaries required for preparing sterile injectables, tablets or gelatin capsules.

Examination of the process chemistry for amonafide readily illustrates the shortcomings of the prior art and the adverse properties of the resulting mineral salts. Among the synthetic approaches described in the patent and professional literature, the common denominator requires acylation of 1-amino-2,2-N,N-dimethylamino ethylene diamine, or its similarly substituted homologs, with a polycyclic, substituted aryl anhydride as shown in FIG. 1. Thus for amonafide, in accordance with the method of Brana and Sanz (Eur. J. Med. Chem 16:207, 1981) compounds I and II in FIG. 1 are combined in ethanol to afford a precipitate of mitonafide, which must then be recrystallized multiple times from a larger volume of ethanol to be freed of tarry-black or brown by-products.

While the acylation may be conducted at a concentration of 1 gram of precursor anhydride in 25 ml of solvent, recrystallization of mitonafide requires three recrystallizations at a concentration of 1 gram in 75 ml to afford light cream colored material, free of tarry substances and exhibiting a constant melting. Although the initial yields according this process range within 60-80%, subsequent purification reduces the net yield to 30% of material with sufficient purity for subsequent conversion into a pharmaceutically acceptable end-product.

These isolation and purification conditions also apply to the synthesis of mitonafide analogs in toluene followed by precipitation with excess gaseous hydrochloric acid, as described by Zee-Cheng and Cheng (U.S. Pat. No. 4,665,071; 1987). The mitonafide hydrochloride, of unspecified stoichiometry and hydration, obtained by this reaction is a reddish brown precipitate, containing 12% by weight of tar with no differential solubility between water and alcohol, again requiring multiple recrystallizations to afford a hydrochloride salt material of suitable quality for pharmaceutical use.

Those skilled in the art will also recognize that salt formation of mitonafide as an isolation step as taught by U.S. Pat. No. 4,665,071 (1987) cannot be adopted for use with organic acids, as many of the latter are known to be insoluble in toluene, and similar non-polar solvents, even at reflux.

Brana and associates (U.S. Pat. No. 5,420,137; 1995) fail to describe the properties of the precursor mitonafide nor do they describe the method of hydrogenation to the resulting amonafide free base, congeneric with Stucture IV in FIG. 1. However, in Spanish Patent 533,542 (1983), a method for the industrial production specifically of amonafide, the authors indicate that nitro reduction of the precursor mitonafide free base is effected with 10% palladium-on-carbon (Pd/C) via transfer hydrogenation in the presence of excess hydrazine under refluxing ethanolic conditions. This procedure is also summarized as the preferred approach in the chemical review literature by the same authors (Ars Pharmaceutica 36:377-415, 1995).

Those skilled in the art would recognize that such an approach could not be practiced if the mitonafide precursor were composed of a pre-formed acid salt. Under such circumstances, one might reasonably expect that the hydrazine donor reagent would be neutralized by ion exchange and become unavailable as a substrate for diimide formation, which is the active reducing species catalyzed by Pd/C.

The catalytic hydrogenation of aryl nitro compounds to the corresponding substituted anilines is usually practiced in ethanol, mixtures of ethanol and water, or in the so called universal solvents (e.g. dimethyformamide and dimethylacetamide) which are resistant to hydrogenation. See, for example, P. N. Rylander (Catalytic Hydrogenation in Organic Synthesis, New York: Academic Press, 1979) and M. Freifelder (Practical Catalytic Hydrogenation, New York: Wiley, 1971) which teach that the solubility of aryl nitro compounds in general precludes use of water as the hydrogenation medium. These experts also indicate that the preferred source of protons to effect suppression of the imine and oxime by-products of incomplete hydrogenation is achieved by admixture of the substrate with hydrochloric acid. Use of organic acids, such as acetic acid or formic acid, has been described, but with the caveat that dehydrative acylation will occur, thus affording the corresponding N-acyl aryl-amines as yield-lowering contaminants.

There is a need in new amonafide salts and in developing synthetic methods for generating same.

### SUMMARY OF THE INVENTION

The instant invention is based on a discovery that organic carboxylic acid salts of amonafide, and, in particular, salts of hydroxy acids, have properties that are unexpectedly superior to the properties of inorganic salts.

In one embodiment, the present invention is an organic hydroxy acid salt of amonafide.

In another embodiment, the present invention is a pharmaceutical composition comprising a pharmaceutically acceptable carrier or diluent and an organic hydroxy acid salt of amonafide.

In another embodiment, the present invention is a method of treating a subject with cancer selected from the group consisting of breast cancer, colon cancer, lung cancer, prostate cancer and leukemia, comprising the step of administering to the subject an effective amount of an organic hydroxy acid salt of amonafide.

Practical advantages of using organic carboxylic acid salts of amonafide are numerous. The resulting aralkyl naphthalimide salts show water solubilites as high as 1:1 by proportional admixture in contrast to the mono or divalent salts of hydrochloric, methanesulfonic, or of other mineral acids, whose solubilities fall below 10% by weight. Bulk processing is facilitated for purposes of industrial synthesis, filtration, purification, and dispensing of dosage units prior to sterile filtration and lyophilization. In dry form, these organic carboxylic acid salts show higher bulk density, porosity and compaction than their analogs mineral acid salts, while presenting lower hygroscopicity. Thus, they are more suitable for processing by direct pressing, rather than solely by granulation or agglomeration.

Furthermore, the data presented in Example 6 demonstrates that (1) organic acid salts of amonafide as a group exhibited higher mean yield and mean purity than a group of their inorganic counterparts, independently of the method of preparation; (2) hydroxyacid salts of amonafide as a group exhibited higher mean yield and mean purity than the entire group of organic acids or the group of mineral acids, independently of the method of preparation; (3) reaction pot yields of all hydroxyacid salts produced according to the inventive method of the instant application but citric acid salt are individually better than yield of the best mineral acid salt (hydrochloric), produced by the same method; reaction pot purities of all hydroxyacid salts produced according to the inventive method of the instant application but citric acid salt are individually at least as good (within the standard deviation) as the yield of the best mineral acid salt (hydrochloric), produced by the same method (Tables 9B and 9C, FIGS. 11B and 11C); (4) after one recrystallization of the reaction pot products produced by the inventive method of Example 1, individual yields of all organic acid salts are better than the best mineral acid salt yield (sulfuric); individual purities of all organic acid salts but malonic are better than the best mineral acid salt purity (hydrochloric, Tables 10A and 10B, FIGS. 13A and 13B); (5) the individual purities of all hydroxyacid salt prepared by the method of Zee-Chang are higher than purity of the best of the mineral acid salt (methanesulfonic, Tables 11A and 11B, FIGS. 15A and 15B).

### BRIEF DESCRIPTION OF THE DRAWINGS

FIG. 1 shows synthetic Schemes (I) and (II). Scheme (I) illustrates the synthesis of amonafide malate salt by the method disclosed herein. Many other organic carboxylic acid salts of the amonafide structural skeleton can be prepared by the method disclosed herein. Scheme (II) illustrates the conceptual steps of amonafide salt synthesis by the method of Example 2.

FIG. 2 is a graph showing the percent net cell growth of cancer cell from the cell lines H460 (♦), SF268 (▪) and MCF7 (▴) in the presence of varying concentrations of amonafide malate. The concentration of amonafide malate is given in μM.

FIG. 3 is a graph showing the percent net cell growth of breast cancer cell from the cell lines MCF-7 (♦), BT474 (∘), MDA-231 (▪), T47D (□) and SKBr3 (▴) using Sulforhodamine B analysis in the presence of varying concentrations of amonafide malate. The concentration of amonafide malate is given in μM.

FIG. 4 is a graph showing the percent net cell growth of colorectal cancer cell from the cell lines HT29 (♦), HCT116 (▪) and COLO205 (▴) using Sulforhodamine B analysis in the presence of varying concentrations of amonafide malate. The concentration of amonafide malate is given in μM.

FIG. 5 is a graph showing the percent net cell growth of lung cancer cell from the cell lines H460 (♦) and H23 (▪) using Sulforhodamine B analysis in the presence of varying concentrations of amonafide malate. The concentration of amonafide malate is given in μM.

FIG. 6 is a graph showing the percent net cell growth of lung cancer cell from the cell lines H460 (♦), H23 (▪) and A549 (▴) using MTT analysis in the presence of varying concentrations of amonafide malate. The concentration of amonafide malate is given in μM.

FIG. 7 is a graph showing the percent net cell growth of prostate cancer cell from the cell lines DU-145 (♦), PC-3 (▪) and LNCaP (▴) using Sulforhodamine B analysis in the presence of varying concentrations of amonafide malate. The concentration of amonafide malate is given in μM.

FIG. 8 is a graph showing the percent growth inhibition by amonafide malate of MCF-7, COLO205 and PC3 tumors in mice. Amonafide malate was administered intraperitoneally (IP) or subcutaneously (SC).

FIG. 9 is a graph showing in vivo percent growth inhibition of intraperitoneally implanted solid tumor by amonafide malate in mice. Cell lines used were MCF-7, MDA-231, H23 and COLO205. Amonafide malate was administered intraperitoneally twice-daily at either 15 mg/kg or 29 mg/kg.

FIG. 10 is a graph showing in vivo percent growth inhibition of intraperitoneally implanted solid tumor by amonafide malate in mice. Cell lines used were MCF-7, MDA-231, H23 and COLO205. Amonafide malate was administered intraperitoneally twice-daily at either 15 mg/kg or 29 mg/kg.

FIG. 11A presents Table 9A. Table 9A shows the results of measurements of recrystallization yields and purities of mineral (inorganic) salts of amonafide synthesized according to the method of Example 1.

FIG. 11B presents Table 9B. Table 9B shows the results of measurements of recrystallization yields and purities of organic hydroxy acid salts of amonafide synthesized according to the method of Example 1.

FIG. 12A presents Table 10A. Table 10A shows the results of measurements of yields and purities of organic acid salts of amonafide synthesized according to the method of Zee-Cheng, as described in Section V of Example 3 of the instant specification.

FIG. 12B presents Table 10B. Table 10B shows the results of measurements of yields and purities of mineral (inorganic) acid salts of amonafide synthesized according to the method of Zee-Cheng, as described in Section V of Example 3 of the instant specification.

### DETAILED DESCRIPTION OF THE INVENTION

The present invention is based on a discovery that the early incorporation of organic acids into the synthetic elaboration of amonafide and its aminoalkyl analogs moieties permits more rapid isolation and purification of intermediates, higher concentrations of reactants during the synthetic process, and more desirable properties, such as bulk density, flocculence and compressibility.

Furthermore, the resulting organic salts show higher solubilities in water as well as in osmotically balanced electrolyte solutions, which otherwise would be incompatible via common ion effects with inorganic, mineral acid salts such as the hydrochlorides and methylsulfonates used routinely heretofore. Moreover, organic acid salts of the present invention retain a higher degree of amphiphilic compatibility both with protic and aprotic solvents of varying polarity, thereby affording a broader range of crystallizing conditions for purposes of purification and isolation than would be afforded by the corresponding mineral acid salts.

The present invention is directed to organic carboxylic acid salts of amonafide and organic carboxylic acid salts of amonafide derivatives and precursors represented by Structural Formula (I): 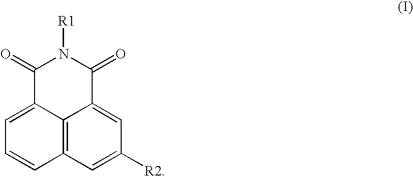
In Formula (I):

R1 is —(CH₂)ₙN⁺HR3R4 X⁻ or R1 is —(CH₂)ₙN⁺HR3R4 X⁻ or —(CH₂)ₙNR3R4 when R2 is —N⁺HR6R7.

R2 is —OR5, halogen, —NR6R7, —N⁺HR6R7 sulphonic acid, nitro, —NR5COOR5, —NR5COR5 or —OCOR5.

R3 and R4 are independently H, C1-C4 alkyl group or, taken together with the nitrogen atom to which they are bonded, a non-aromatic nitrogen-containing heterocyclic group.

Each R5 is independently —H or a C1-C4 alkyl group.

R6 and R7 are independently H, a C1-C4 alkyl group or, taken together with the nitrogen atom to which they are bonded, a non-aromatic nitrogen-containing heterocyclic group.

n is an integer from 0-3.

X⁻ is the carboxylate anion of an organic carboxylic acid compound. Examples of suitable organic carboxylic acids are provided below.

Preferably in Structural Formula (I), n is 2; R3 and R4 are the same and are —H, —CH₃ or —CH₂CH₃; and R2 is —NO₂, —NH2 or —NH₃⁺X⁻. More preferably, n is 2; R3 and R4 are —CH₃; and R2 is —NO₂, —NH₂ or —NH₃+X⁻. Suitable values for X⁻ are provided below.

Most preferably, the present invention is directed to organic carboxylic acid salts of amonafide and methods of preparation thereof. The structure of amonafide is represented by Structural Formula (II):

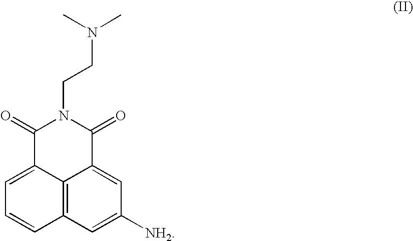

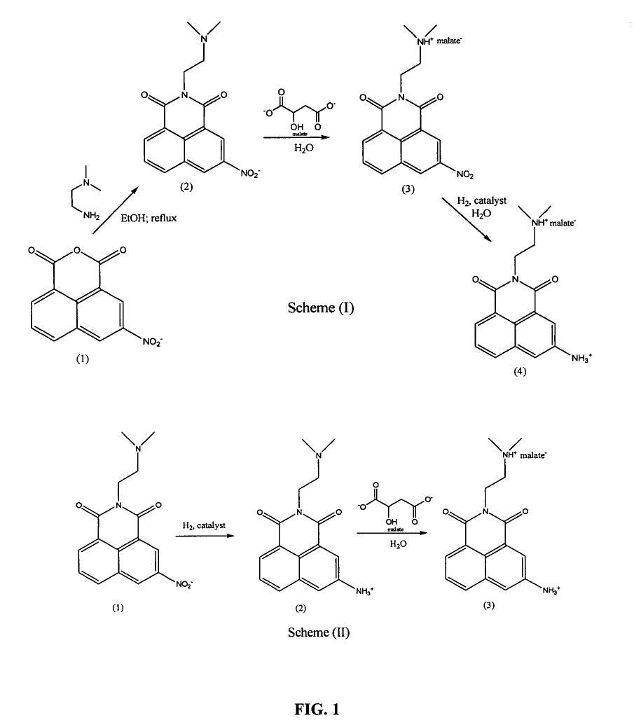

The present invention is also directed to methods of preparing organic acid salts of compounds of Formula (I) in water according to synthetic Scheme (I) depicted in FIG. 1. In Scheme (I), organic acid salt is exemplified by malate and compounds of formula (I) are exemplified by mitonafide (2), mitonafide salt (3) and amonafide salt (4).

The compounds disclosed herein with two amine groups, including amonafide salts, can be monovalent, meaning that one of the amine groups is protonated, or divalent, meaning that both amine groups are protonated. A divalent compound can be protonated by two different monocarboxylic acid compounds (i.e., the two Xs in Structural Formula (I) represent two different monocarboxylic acid compounds), by two molar equivalents of the same monocarboxylic acid compound (i.e., the two Xs in Structural Formula (I) each represent one molar equivalent of the same monocarboxylic acid compound), or by one molar equivalent of a dicarboxylic acid compound (i.e., the two Xs in Structural Formula (I) together represent one dicarboxylic acid compound). Alternatively, three molar equivalents a divalent compound are protonated by two molar equivalents of a tricarboxylic acid compound. All of these possibilities are meant to be included within Structural Formulas (I) and (II) above.

An organic carboxylic acid compound is an organic compound having one or more carbon atoms and a carboxylic acid functional group. Suitable organic carboxylic acid compounds for use in preparing the compounds of the present invention are water soluble (typically a water solubility greater than 20% weight to volume), produce water soluble salts with aryl amines and alkyl amines and have a pKa>2.0. Included are aryl carboxylic acids, aliphatic carboxylic acids (typically C1-C4), aliphatic dicarboxylic acids (typically C2-C6), aliphatic tricarboxylic acids (typically C3-C8) and heteroalkyl carboxylic acids. An aliphatic carboxylic acid can be completely saturated (an alkyl carboxylic acid) or can have one or more units of unsaturation. A heteroalkyl carboxylic acid compound is an aliphatic carboxylic acid compound in which one or more methylene or methane groups are replaced by a heteroatom such as O, S, or NH. Examples of heteroalkyl carboxylic acid compounds include a C1-C5 heteroalkyl monocarboxylic acid compound (i.e., a C2-C6 alkyl monocarboxylic acid compound in which one methylene or methane group has been replaced with O, S or NH) and C3-C8 a heteroalkyl dicarboxylic acid compound (i.e., a C2-C7 alkyl dicarboxylic acid compound in which one methylene or methane group has been replaced with O, S or NH).

An aliphatic carboxylic acid compound can be straight or branched. An aliphatic carboxylic acid can be substituted (functionalized) with, one or more functional groups. Examples include a hydroxyl group (e.g., a hydroxy C2-C6 aliphatic monocarboxylic acids, a hydroxy C3-C8 aliphatic dicarboxylic acid and a hydroxy C4-C10 hydroxy aliphatic tricarboxylic acid), an amine (e.g., an amino C2-C6 aliphatic monocarboxylic acid, an amino C3-C8 aliphatic dicarboxylic acid and an amino C4-C10 aliphatic tricarboxylic acid), a ketone (e.g., a keto C2-C6 aliphatic monocarboxylic acid, a keto C3-C8 dicarboxylic acid or a keto C4-C10 tricarboxylic acid) or other suitable functional group.

Examples of suitable organic acids are:

saturated aliphatic monocarboxylic acids such as formic acid, acetic acid or propionic acid;

unsaturated aliphatic monocarboxylic acids such as 2-pentenoic acid, 3-pentenoic acid, 3-methyl-2-butenoic acid or 4-methyl-3-pentenoic acid;

functionalized acids such as hydroxycarboxylic acids (e.g. lactic acid, glycolic, pyruvic acid, mandelic acid);

ketocarboxylic acids (e.g. oxaloacetic acid and alpha-ketoglutaric acid);

amino carboxylic acids (e.g. aspartic acid and glutamic acid);

saturated aliphatic dicarboxylic acids such as malonic acid, succinic acid or adipic acid;

unsaturated aliphatic dicarboxylic acids such as maleic acid or fumaric acid;

functionalized di- and tricarboxylic acids such as malic acid, tartaric acid, citric acid gluconic acid.

aryl carboxylic acids having sufficient water solubility, e.g., e.g., 4-hydroxybenzoic acid, salicylic acid, anthranilic acid, anisic acid and vanillic acid.

Non-aromatic nitrogen-containing heterocyclic rings are non-aromatic nitrogen-containing rings which include zero, one or more additional heteroatoms such as nitrogen, oxygen or sulfur in the ring. The ring can be five, six, seven or eight-membered. Examples include morpholinyl, thiomorpholinyl, pyrrolidinyl, piperazinyl, piperidinyl, azetidinyl, azacycloheptyl, or N-phenylpiperazinyl.

Referring to FIG. 1, hydrogenation of salt (3), exemplified in synthetic Scheme (I) by a salt of mitonafide, is carried out under a hydrogen atmosphere at pressures between 5 and 50 pounds per square inch (psi), preferably between 13 and 17 psi. A hydrogenation catalyst is required, for example Pd/C, Pt/C, PtO₂, Raney Nickel and activated elemental iron or zinc. After hydrogenation, a monovalent compound is obtained. The corresponding divalent compound can be obtained by reacting the product with an additional equivalent of the same or different carboxylic acid compound.

Salt (3) of Scheme (I), can be prepared by reacting the corresponding free base (2) (exemplified in Scheme (I) by mitonafide free base), with an organic carboxylic acid compound. Preferably, the resulting product is crystallized before hydrogenating.

Free base (2) can be prepared by reacting H₂N(CH₂)ₙNR3R4 with compound (1), exemplified in Scheme (I) by 3-nitro-1,8-nitronaphthalic anhydride. Specific conditions for carrying out this reaction are described in U.S. Pat. No. 4,204,063, the entire teachings of which are incorporated herein by reference. Synthetic Scheme (I) exemplifies a reaction of nitro-1,8-nitronaphthalic anhydride with H₂N(CH₂)₂NH₂ to produce mitonafide.

The compounds disclosed herein are useful for the treatment of a subject. A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like). The compounds of the present invention can be used to treat a broad spectrum of cancers, including carcinomas, sarcomas and leukemias. Examples of carcinomas, including adenocarcinomas that can be treated using the compounds of the present invention are breast, colon, lung, kidney and prostate cancers. An example of sarcomas that can be treated using the compounds of the present invention are gliomas. Examples of leukemias that can be treated using the method include Acute Myelogenous Leukemia (AML), Chronic Myelogenous Leukemia (CML), Acute Lymphocytic Leukemia (ALL) and Chronic Lymphocytic Leukemia (CLL). Preferably, the cancers that are treated using the compounds of the present invention are breast, colorectal, lung and prostate cancers.

An “effective amount” is the quantity of compound in which a beneficial clinical outcome is achieved when the compound is administered to a subject with a multi-drug resistant cancer. A “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of cancer. The skilled artisan will be able to determine appropriate dosages depending on these and other factors. Effective amounts of the disclosed compounds for therapeutic application typically range between about 0.35 millimoles per square meter of body surface area (mmole/msq) per day and about 2.25 millimoles per square meter of body surface area (mmole/msq) per day, and preferably between 1 mmole/msq and 1.5 mmole/msq on five day cycles by intravenous infusion.

The disclosed compounds are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), or rectally, depending on the type of cancer to be treated. Oral or parenteral administration are preferred modes of administration.

The disclosed compounds can be administered to the subject in conjunction with an acceptable pharmaceutical carrier as part of a pharmaceutical composition for treatment of cancer. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

### EXEMPLIFICATION

The method of the invention is illustrated in more detail by the following examples which are not intended to be limiting in any way. The course of the organic acid salt formation of amonafide and analogs thereof can be followed via the determination of the products formed by means of chromatography and NMR spectroscopy. The formed salts are further characterized by mass spectrometry and by elementary analysis.

### Example 1

### Direct Synthesis of Intercalator Drug Amonifide as an Organic Acid (L-malate) Salt by the Method of the Present Invention

1. Preparation of mitonafide malate (III, FW 447.42)

Referring to FIG. 1, preparation of mitonafide malate (3) was carried out according to Scheme (I).

Reactants:

(A) 3-nitro-1,8-nitronaphthalic anhydride, (FW 243.18, CAS 3027-38-1, purity 99%, ACROS, cat. # 27873-0250);

(B) N,N-dimethylethylenediamine (FW 88.15, CAS 108-00-9, purity 99%, ACROS cat. # 11620-100);

(C) L-malic acid (FW 134.09, CAS 97-67-6, purity 99% ACROS cat. # 15059-1000

Synthetic Procedure:

100 gr. (0.41 mol, 1 eq.) of the anhydride (A) were combined with 1300 ml of anhydrous ethanol in a 3 L 3-neck round bottom flask fitted with an adding funnel and mechanical paddle stirrer. While vigorously stirring the suspension, a solution of 40 gr (0.45 mol, 1.1 eq.) of the diamine (B) in 100 ml of anhydrous ethanol was added as a rapid drip. Stirring was continued for 12 hours (overnight), and thereafter the mixture was brought to reflux for 1 hour.

Upon cooling to an internal temperature of 80° C., a pre-warmed (also to 80° C.) solution of 60 gr. (0.42 mol, 1.09 eq.) of L-malic acid in 100 ml of ethanol was added in one portion to the reaction flask and stirring continued for 3 hours. Stirring was stopped and when the reaction reaches room temperature, the crude mitonafide malate was recovered by filtration. The solids were resuspended in 1 liter of anhydrous isopropanol, refiltered, and rinsed with diethyl ether. They were then transferred to a drying dish, triturated mechanically and vacuum desiccated in a drying oven at 0.1 Torr with heating to 30° C. for 12 hours.

A tan solid (160 gr., 87% yield) was obtained, mp 160-162° C., with a proton NMR spectrum (in perdeuterated acetic acid, 80 MHz) conforming to theory: malate-CH₂, 2.8 ppm, asymmetric doublet, 2H; N,N—(CH₃)₂, 3.1 ppm, singlet, 6H; imido-N—CH₂, 3.7 ppm, degenerate triplet, 2H; amino-N—CH₂ and malate-CH, 4.6 ppm, multiplet, 3H; aryl-CH, 8-9.3 ppm, two apparent triplets and doublet, 5H; OH, 11.4 ppm, singlet, 3H.

Material prepared in this manner was sufficiently pure for use in subsequent synthetic steps.

Alternatively, for analytical and biological samples, recrystallization was achieved by suspending the product in aqueous ethanol (water/ethanol 1/5 v/v per gram of mitonafide malate), heating to boil and removing any insolubles by hot filtration. Upon cooling, the mass was filtered, rinsed with diethyl ether and vacuum desiccated to afford light tan, birefringent plates (140 gr, 76% overall yield), mp 163-164° C., homogenous by HPLC, under the conditions shown in Table 1.
2. Preparation of amonafide malate (FW 417.42)

Referring to FIG. 1, preparation of amonafide malate (4) was carried out according to synthetic Scheme (I).

Synthetic Procedure:

A solution of 134 gr. (0.3 mole) mitonafide malate was suspended in 1 liter of deionized, degassed water under an argon blanket in a Parr hydrogenation pressure bottle. 1.4 gr. of 10% Pd/C are added, and the mixture was then evacuated and purged with hydrogen gas (three times), then connected to a Parr apparatus and pressurized with hydrogen gas to 15 psi. The reaction was vigorously shaken at room temperature becoming a yellow solution instead of a tan colored suspension within two hours. It was left to hydrogenate for an additional 12 hours (overnight).

After evacuative removal of the hydrogen headspace and replacement with nitrogen, the reaction mixture was stirred with 40 gr. of activated carbon, warmed to 50° C., and passed in a Buchner funnel through filter paper overlayed with pre-washed Celite filtration aid. The filtrate was concentrated in a 2 liter round bottom flask, under reduced pressure in a rotary evaporator with a heating bath thermostated to 50° C. When a thin crust had begun to form along the meniscus of the syrupy concentrate, which weighed between 240-250 gr, the flask was refrigerated (4° C.) for 12 hrs (overnight) to permit crystallization.

The resulting mustard yellow crystals and mother liquor were triturated with isopropanol, which was added in portions to a total volume of 1 liter. After an additional 2 hrs. of refrigeration, the suspension was filtered, washed with isopropanol and diethyl ether to afford amonafide malate, 113 gr. (90%), as a mustard yellow powder, mp 182-184° C., after desiccation in a heated vacuum oven (40-50° C., 0.5 Torr, 14 hrs.). The proton NMR spectrum (in perdeuterated acetic acid, 80 MHz) conformed to theory: malate-CH₂, 2.8 ppm, asymmetric doublet, 2H; N,N—(CH₃)₂, 3.1 ppm, singlet, 6H; imido-N—CH₂, 3.7 ppm, degenerated triplet, 2H; amino-N—CH₂ and malate-CH, 4.6 ppm, multiplet, 3H; aryl-CH, 7.4-8.2 ppm, two apparent multiplets, 5H; OH, 11.4 ppm, singlet, 5H.

For comparative purposes, between and among organic salts prepared in this manner and for the purposes of subsequent Example 3, Table 3, crude reaction products were recrystalized from water/ethanol (1.5/4 v/v per gram of amonafide malate) using the minimum amount of solvent to effect dissolution at boil.

Alternatively, for analytical and bioassay samples, recrystallization was achieved by dissolving the product in water (1/4 w/v per gr. of amonafide malate) and admixing with a hot mixture of 1/1 isopropanol/methanol (1/6 w/v per gr. of amonafide malate), heating to boil and removing any insolubles by hot filtration. Upon cooling first to room temperature and then upon chilling for 12 hrs. (0-4° C.), the mass was filtered, rinsed with isopropanol followed by diethyl ether and vacuum desiccated to afford 90 gr. of mustard yellow, rhomboid crystals (85 gr., 68% overall yield), mp 184-185° C., homogenous by HPLC, under the conditions shown in Table 1.

TABLE 1
HPLC OF MITONAFIDE AND AMONAFIDE MALATE

| | Mobile | Flow Rate | Col Temp | Rt | |
|---|---|---|---|---|---|
| Column* | Phase** | ml/min | (° C.) | AMFm | MiTm |
| Nova Pak C18 | | | | | |
| 4 um 60A° | 17/55/28 | 1.0 | 30 | >30 | NA |
| 3.9 × 150 mm. | 30/35/35 | 2.5 | 40 | 10.2 | 13.2 |
| | 40/25/35 | 2.5 | 40 | 5.1 | 4.7 |
| Xterra MS C18 | 17/55/28 | 0.8 | 40 | 1.4 | 3.1 |
| 3.5 uM | 17/55/28 | 0.8 | 30 | 2.3 | NA |
| 3.0 × 150 mm. | | | | | |

*End-capped (shielded) columns should be used
**CH₃CN, H₂O, MeOH v/v/v
AMFm = amonafide malate
MiTn = mitonafide malate

### Example 2

### Synthesis of Amonifide Organic Carboxylic Acid Salts by Titration According to the Method of U.S. Pat. No 5,183,821

A panel of salts were readily prepared in semi-automated fashion. It should be understood that any analog or congener thereof, with similar aralkylamine derived basicity properties, would be equally suitable as an exemplar.

Scheme (II), FIG. 1, illustrates the conceptual steps of amonafide salt synthesis in this example.

Referring to Scheme (II), a stock solution of amonafide free base (2) was first dispensed into individual reaction vials so as to provide a defined amount of basic substrate. It was then titrated with a second solution containing one stoichiometric equivalent of an appropriate organic carboxylic acid, whose acidity is consistent with an aqueous pKa value of not less than 3. The resulting mixture was warmed in order to effect complete dissolution and neutralization of the species reacting ionically, and allowed to deposit the resulting salts as products upon cooling. These solutions may also be concentrated, prior to cooling, in order to optimize the reaction yield. However, for optimal results the reaction solvent in this manipulation should be selected so that the reactants are individually more soluble than their ionic combination.

Referring to Scheme (II), free base material (2) was synthesized according to Brana et al in U.S. Pat. No. 5,183,821. An aliquot was dissolved in boiling anhydrous ethanol at a concentration of 1 gram per 20 ml. 10 ml of solution prepared in this manner contains 1.765 mMol of material and would, therefore, become neutralized by an equivalent amount of an appropriate organic carboxylic acid in order to afford a monovalent organic salt. Since amonafide is divalent, in theory, it can also be titrated with two equivalents of organic acid to afford a divalent salt. Thus, the number of acid equivalents that can be added should at least match the calculated minimum number of basic equivalents in the intercalator free drug and not exceed the maximum such number.

For the purpose of this example, however, salt formation has been restricted to monoequivalents, and, therefore, solutions of organic carboxylic acids containing 1.765 mMol in 10-20 ml of water at boil were prepared and added individually to each of several replicate 100 ml portions of the drug free base at the concentration and volume just described. After bringing the mixed solutions again up to boil, to insure complete dissolution of all ingredients, they were then left to cool at room temperature and refrigerated overnight, whereupon the resulting crystalline salts were harvested by filtration, dried by rinsing with diethyl ether, and desiccated under vacuum.

Table 2 provides a listing of the organic carboxylic acids that were used to titrate amonafide in order to produce the corresponding crystalline monovalent salt. Salts obtained in this manner may be characterized variously by chromatography for chemical homogeneity, and by NMR or mass spectrometry for purposes of structural characterization, as described herein above in Example 1. Characterization by elemental composition also affords a convenient method for identity verification. As shown in Table 2, the observed compositions in Part I and the theoretical elemental compositions in Part II are closely matched, demonstrating that the reaction products constitute an equimolar addition of each reactant, as would be anticipated for any such monovalent adducts of an organic base and an organic acid.

TABLE 2
AMONAFIDE SALTS COMPOSITION OF ORGANIC SALT
Part I

Observed

| Acid | C | H | N | O |
|---|---|---|---|---|
| A) succinic | 59.54 | 5.89 | 10.13 | 24.44 |
| B) maleic | 60.37 | 5.10 | 10.23 | 24.30 |
| C) fumaric | 59.89 | 5.52 | 10.20 | 24.39 |
| D) citric | 54.87 | 5.78 | 8.30 | 31.05 |
| E) L-tartaric | 56.27 | 4.95 | 9.23 | 29.55 |
| F) L-aspartic | 56.80 | 6.02 | 13.78 | 23.40 |
| G) pyruvic | 62.45 | 5.34 | 11.00 | 21.21 |
| H 2-oxoglutaric | 58.12 | 5.30 | 10.40 | 26.18 |

Part II

Calculated

| Acid | C | H | N | O |
|---|---|---|---|---|
| A) succinic | 59.84 | 5.78 | 10.47 | 23.91 |
| B) maleic | 60.14 | 5.30 | 10.52 | 24.03 |
| C) fumaric | 60.14 | 5.30 | 10.52 | 24.03 |
| D) citric | 55.58 | 5.30 | 8.84 | 30.29 |
| E) L-tartaric | 55.42 | 5.35 | 9.69 | 29.53 |
| F) L-aspartic | 57.69 | 5.81 | 13.45 | 23.05 |
| G) pyruvic | 61.45 | 5.70 | 11.31 | 21.54 |
| H 2-oxoglutaric | 58.74 | 5.40 | 9.79 | 26.08 |

Further confirmation of structural identity and purity was also obtained in each instance by NMR analysis in perdeutero acetic acid, the salt of amonafide and succinic acid being representative, in so far as it is a comparable analog to the malate salt prepared in Example 1. Thus, the salt shown prepared according to Table 2, Part I, entry A, as the monoequivalent combination of amonafide and succinic acid showed a proton NMR spectrum (in perdeuterated acetic acid, 80 MHz) conforming to theory: succinate-CH₂, 2.7 ppm, singlet, 6H; N,N—(CH₃)₂, 3.1 ppm, singlet, 6H; imido-N—CH₂, 3.7 ppm, degenerated triplet, 2H; amino-N—CH₂, 4.6 ppm, degenerated triplet, 2H; aryl-CH, 7.4-8.3 ppm, two apparent multiplets, 5H; OH, 11.4 ppm, singlet, 4H.

Although this example illustrates the use of several chiral molecules, as for example in entries E-F, it follows that suitable results for purposes of salt formation can be obtained with the corresponding racemic form or alternate antipodes of such acids. Thus, the selection of the L-enantiomers in this instance should not be taken as a restriction of the teaching; but, rather, as a case in point that would be understood by those practiced in the art to be the most commonly available such organic carboxylic acid forms suitable for purposes of biological experimentation.

### Example 3

### Organic Salts of Amonifide are More Readily Purified than HCl or Methanesulfonic Acid Salts of Amonifide Independent of Method of Synthesis

I. Direct Synthesis Amonafide Monohydrochloride Salt by the Method of Example 1
1. Preparation of mitonafide monohydrochloride (FW 349.82)

Referring to FIG. 1, preparation of mitonafide monohydrochloride (3) was carried out according to Scheme (I).

Reactants:

(A) 3-nitro-1,8-nitronaphthalic anhydride, (FW 243.18, CAS 3027-38-1, purity 99%, ACROS, cat. # 27873-0250);

(B) N,N-dimethylethylenediamine (FW 88.15, CAS 108-00-9, purity 99%, ACROS cat. # 11620-100);

(C) 6.0 N Hydrochloric acid (FW 36.45 CAS 7647-01-0, purity 99.9% ACROS cat. # 61327-0010

Synthetic Procedure:

10 gr. (0.041 mol, 1 eq.) of the anhydride (A) were combined with 130 ml of anhydrous ethanol in a 0.3L 3-neck round bottom flask fitted with an adding funnel and mechanical paddle stirrer. While vigorously stirring the suspension, a solution of 4 gr (0.045 mol, 1.1 eq.) of the diamine (B) in 10 ml of anhydrous ethanol was added as a rapid drip. Stirring was continued for 12 hours (overnight), and thereafter the mixture was brought to reflux for 1 hour.

Upon cooling to an internal temperature of 80° C., a pre-warmed (also to 80° C.) solution of 7.5 ml of 6.0 N HCl (0.045 mol, 1.1 eq.) and 2.5 ml of ethanol was added in one portion to the reaction flask and stirring continued for 3 hours. Stirring was stopped and when the reaction reaches room temperature, the crude mitonafide monohydrochloride was recovered by filtration. The solids were resuspended in 0.1 liter of anhydrous isopropanol, refiltered, and rinsed with diethyl ether. They were then transferred to a drying dish, triturated mechanically and vacuum desiccated in a drying oven at 0.1 Torr with heating to 30° C. for 12 hours.

A tan solid (11.9 gr., 83% yield) was obtained, which on recrystallization from water ethanol, 2:1 v/v, afforded 8.3 gr, mp 180-183° C. homogenous by HPLC, under the conditions described in Example I.
2. Preparation of amonafide monohydrochloride (FW 319.79)

Preparation of amonafide monohydrochloride (4) was carried out according to synthetic Scheme (I).

A solution of 10.5 gr. (0.03 mole) mitonafide monohydrochloride was suspended in 0.1 liter of deionized, degassed water under an argon blanket in a Parr hydrogenation pressure bottle. 0.15 gr. of 10% Pd/C are added, and the mixture was then evacuated and purged with hydrogen gas (three times), then connected to a Parr apparatus and pressurized with hydrogen gas to 15 psi. The reaction was vigorously shaken at room temperature becoming a yellow solution instead of a tan colored suspension within two hours. It was left to hydrogenate for an additional 12 hours (overnight).

After evacuative removal of the hydrogen headspace and replacement with nitrogen, the reaction mixture was stirred with 4 gr. of activated carbon, warmed to ° C., and passed in a Buchner funnel through filter paper overlayed with pre-washed Celite filtration aid. The filtrate was concentrated in a 0.25 liter round bottom flask, under reduced pressure in a rotary evaporator with a heating bath thermostated to 50° C. When a thin crust had begun to form along the meniscus of the syrupy concentrate, which weighed between 23-24 gr, the flask was refrigerated (4° C.) for 12 hrs (overnight) to permit crystallization.

The resulting mustard yellow crystals and mother liquor were triturated with isopropanol, which was added in portions to a total volume of 0.1 liter. After an additional 2 hours of refrigeration, the suspension was filtered, washed with isopropanol and diethyl ether to afford 7.9 gr. (81%), as a mustard yellow powder, mp 184-88° C., after desiccation in a heated vacuum oven (40-50° C., 0.5 Torr, 14 hrs.). However, the material was non-homogenous by HPLC, under the conditions shown in Table 1, revealing a main fraction consisting 91% of the peak areas and at last 4 additional impurities ranging in areas from 0.5 to 4.5% of the total analyte areas.

Recrystallization was achieved by dissolving the product in water (1/20 w/v per gr.) and admixing with a hot mixture of 1/1 isopropanol/methanol (1/20 w/v), heating to boil and removing any insolubles by hot filtration. Upon cooling first to room temperature and then upon chilling for 12 hrs. (0-4° C.), the mass was filtered, rinsed with isopropanol followed by diethyl ether and vacuum desiccated to afford mustard yellow needles (6.3 gr., 65% overall yield), mp 289-290° C. Chromatographic analysis revealed the recrystallized material to be 97.3% pure and containing an impurity of 1.6%, consisting of the incompletely hydrogenated. A second recrystalization afforded 4.2 (52% overall yield) grams of material with a purity of 99.1%.

As shown in Table 3, the yield and the need for additional recrystallization steps offer a clear contrast to the results of the congeneric synthesis of organic acid salts of amonafide, e.g. amonafide L-malate from mitonafide L-malate. The latter affords purer product in fewer steps, and with a lower solvent volume.
II. Preparation of Organic Salts of Amonafide by the Method of Example 1

Organic Salts of Amonafide were prepared according to Scheme (I), FIG. 1.

Amonafide malate, amonafide fumarate, amonafide maleate, amonafide malonate and amonafide hemisuccinate were prepared according to the method of Example 1. In the case of succinic acid, the organic acid was dispensed as a half-equivalent in order to generate mitonafide hemisuccinate. This method is illustrated schematically in FIG. 1 for amonafide malate. Crude product that was isolated from the hydrogenation reaction mixture was analyzed for purity by high pressure liquid chromatography (HPLC) according to the conditions described in Table 1 above. The crude product was recrystallized from water/ethanol (1.5/4 v/v) using the minimum amount of solvent required to dissolve the crude product at boil, which is shown in the last column of Table 3 for each salt. The recrystallized product was then analyzed for purity by HPLC. The purity for the isolated and recrystallized salts are reported below in Table 3.
III. Preparation of Organic Acid Salts of Amonafide by Either Method of Example 1 or Method of Example 2

As shown in the second column of Table 3 below, fumarate, malate, maleate, malonate and hemi-succinate salts of amonafide were obtained according to the procedures described in Example 1 by in situ formation of the corresponding mitonafide salt, followed by catalytic hydrogenation. Also, the adipate, aspartate, citrate, glycolate, malate, oxoglutarate, pyruvate, salicylate, succinate, and tartrate salts of amonafide were obtained according to the procedures described in Example 2 from the crude amonafide free base prepared as described above in the previous paragraph. The purities of the crude salts were assessed by HPLC according to the conditions described in Table 1 above. The crude salts were then recrystallized from the minimum amount of water/ethanol (1.5/4 v/v) using the minimum amount of solvent required to dissolve the crude product at boil, which is shown in the last column of Table 3 for each salt. The resulting purified salts were again assessed for purity. The purity for the crude and recrystallized salts are shown in Table 3 below.
IV. Preparation of the Hydrochloride Salt and Methanesulfonic Acid Salt of Amonafide the Method of U.S. Pat. No. 5,420,137
1. Preparation of Amonafide Free Base

Amonafide free base was prepared according to procedures disclosed in U.S. Pat. No. 5,183,821 to Brana et al. (See Scheme (II) above.) Exemplary conditions, at 1/100^{th} the reported scale, are as follows: 30 g of 2-(2-dimethylaminoethyl)-5-nitrobenzo[d,e]isoquinoline-1,3-dione and 0.75 gr. of 10% palladium on carbon in 750 milliliters of ethanol were refluxed with stirring until complete dissolution of the nitro compound. Thereupon 40 milliliters of hydrazine hydrate, meeting current commercial specifications of 60% anhydrous hydrazine equivalence, were added slowly over a 30 minute period. When addition was complete, reflux and stirring were continued for 3.5 hours, followed by filtration while still hot. The reaction mixture was left overnight to reach room temperature. The crystalline product was then filtered to afford amonafide free base.
2. Preparation of Amonafide Hydrochloride and Methanesulfonic Salts.

The hydrochloride and methanesulfonic acid salts of amonafide were prepared according to U.S. Pat. No. 5,420,137 (see Scheme (II) above). Exemplary procedures are as follows: 3 g of crude amonafide free base, obtained as described in the previous two paragraphs, above, were dissolved under reflux in 60 ml of virtually anhydrous ethanol. Subsequently 1 ml of 35% (1 equivalent, estimated) strength hydrochloric acid was added dropwise while shaking vigorously. After cooling, the resulting crystals were filtered off and washed with 10 ml of anhydrous ethanol. The methanesulfonic acid salt was obtained using the same procedure, but replacing hydrochloric acid with 0.8 ml of methanesulfonic acid. The purity was assessed by HPLC using the conditions described in Table 1. The isolated products were then recrystallized from water/ethanol (1.5/4 v/v) using the minimum amounts of solvent required to dissolve the crude product at boil, which are shown for each salt in Table 3. The resulting purified salts were again assessed for purity. The results are shown in Table 3 below.
V. Preparation of the Hydrochloride Salt of Amonafide by the Method of Zee-Chang et al., U.S. Pat. No. 4,614,820

Synthetic scheme employed by Zee-Chang et al., U.S. Pat. No. 4,614,820, is similar to Scheme (II), wherein the amonafide free base is catalytically hydrogenated prior to addition of salt.

The hydrochloride salt of amonafide was obtained by under the following exemplary conditions: a mixture of the hydrochloride salt of N-(dimethylaminoethyl)-3-nitro-1,8-naphthalimide and 10% palladium-on-charcoal in water was hydrogenated at room temperature under 40 lbs/in² of hydrogen for one hour. The theoretical amount of hydrogen was absorbed. To the mixture was added 3 ml of concentrated hydrochloric acid. It was then filtered and the filtrate was evaporated to dryness under reduced pressure. The residue was triturated with 30 ml of absolute ethanol and filtered. The resulting solid was then washed with ether and dried. The purity was assessed by HPLC using the conditions described in Table 1. The crude product was then recrystallized from water/ethanol (1.5/4 v/v) using the minimum amount of solvent (40 mL/g) required to dissolve the crude product at boil. The resulting purified salts were again assessed for purity. The results are shown in Table 3 below.
VI. Indirect Synthesis of Amonafide Monohydrochoride via Salt Exchange from Presursor Amonafide Malate
1. Preparation of Amonafide free base.

A batch of amonafide L-malate was prepared in accordance with the method of Example I. The material so obtained was 99.4% chromatographically pure on a 10 gram synthetic scale, consistent with previous findings on the larger scale. The material was then converted to the free base amonafide, as follows: 10 grams of the L-malate salt were dissolved in 100 ml distilled water and titrated with vigorous stirring to pH 7 by the addition of 1/4 concentrated ammonium hydroxide. The large mass of yellow needles that formed was filtered by suction, washed with water, and recrystalized from ethanol to afford 5.8 gr. Of 99.8% chromatographically pure amonafide free base, mp 172° C. (MW 283.3, 93% yield).
2. Preparation of Amonafide Monohydrochloride

The free base was converted into the monohydrochloride by suspending 5 gr in 10 ml of water and adding 1 equivalent of 6.0 normal (2.9 ml) with vigorous stirring and warming. Upon dissolution, 20 ml of boiling ethanol are added slowly to the clouding point, and the mixture allowed to cool and deposit crystals, which are then harvested by filtration at room temperature, rinsed with ethanol and ether, and vacuum dried to afford 5.4 grams of product, mp 292° C. The amonafide monohydrochloride prepared by the method of salt exchange was found to be 99.2% chromatographically pure and free of any detectable N-hydroxylamine. Notwithstanding this level of purity, the amonafide monohydrochloride prepared by salt exchange showed low solubility when included in the test panel shown in Tables 3 and 4.

TABLE 3

| | | Purity | | |
|---|---|---|---|---|
| | | (from | | |
| | | reaction | Purity (after 1× | Solvent |
| Salt | Method | pot) | crystallization) | (ml/g) |
| Amonafide | Example 1 | 92% | 99.3% | 7.5 |
| Malate | | | | |
| Amonafide | Example 2 | 98% | 99.6% | 7.5 |
| Malate | | | | |
| Amonafide HCl | U.S. Pat. No. | 76% | 94% | 40 |
| | 5,420,137 to | | | |
| | Brana et al. | | | |
| Amonafide HCl | U.S. Pat. No. | 74% | 92% | 40 |
| | 4,614,820 to | | | |
| | Zee-Cheng, | | | |
| | et al. | | | |
| Amonafide HCl | Example 1 | 93% | 97.3% | 40 |
| Amonafide HCl | Salt | N/A | 99.2% | 40 |
| | exchange | | | |
| Amonafine | U.S. Pat. No. | 86% | 93% | 25 |
| methanesulfonate | 5,420,137 to | | | |
| | Brana et al | | | |
| Amonafide | Example 2 | 90% | 99.2% | 20 |
| tartrate | | | | |
| Amonafide | Example 2 | 89% | 98% | 25 |
| Adipate | | | | |
| Amonafide | Example 2 | 78% | 98.7% | 30 |
| Aspartate | | | | |
| Amonafide | Example 2 | 92% | 99.1% | 25 |
| Citrate | | | | |
| Amonafide | Example 1 | 76% | 98.9% | 25 |
| Fumarate | | | | |
| Amonafide | Example 2 | 92% | 99.3% | 12.5 |
| glycolate | | | | |
| Amonafide | Example 1 | 70% | 99.2% | 20 |
| maleate | | | | |
| Amonafide | Example 1 | 74% | 97% | 20 |
| malonate | | | | |
| Amonafide 2- | Example 2 | 77% | 98.1% | 30 |
| oxoglutarate | | | | |
| Amonafide | Example 2 | 84% | 99.5% | 20 |
| pyruvate | | | | |
| Amonafide | Example 2 | 87% | 98.7% | 35 |
| salicylate | | | | |
| Amonafide | Example 1 | 88% | 98.7% | 15 |
| hemi-succinate* | | | | |
| Amonafide | Example 2 | 93% | 99.2% | 20 |
| succinate | | | | |
| Amonafide L- | Example 2 | 90% | 99.2% | 20 |
| tartrate | | | | |

*hemisuccinate prepared from mitonafide hemisuccinate, wherein 0.5 equivalents of succinic acid were used in the mitonafide isolation. By contrast, the succinate salt is prepared when admixing one equivalent.

As is evident from the data shown in Table 3, the purity of recrystallized organic acid salts of amonafide was in every case significantly greater than the corresponding recrystallized hydrochloric acid salt and methanesulfonic acid salt. Moreover, in comparing the malate to the HCl moieties, the recrystallization volume for purification of the malate salt was 5 times smaller, indicating both greater solubility in hydroxylic media and also greater ease of handling in a concentrated solution. The glycolate and hemisuccinate salts were also significantly more soluble than either the HCl or methanesulfonic acid salt.

### Example 4

### Amonafide Malate has Anti-Cancer Activity In Vitro

Amonafide Malate was tested for cytotoxic activity in a panel of three cell lines, MCF7 (Breast), H460 (Non-Small Cell Lung Cancer) and SF268 (Glioma). These cell lines are used by the National Cancer Institute: Developmental Therapeutics Branch in their preliminary screening process for antineoplastic agents. Subsequently, Amonafide Malate was tested against a larger panel of breast, lung and colon cell lines

Breast: MCF-7, BT474, MDA-231, T47D and SKBr3

Colon: HT29, HCT116 and COLO205

Lung: H460, H23 and A549

Prostate: DU-145, PC-3 and LNCaP

The following protocol was used:

Cells were grown in RPMI 1640 medium containing 5% fetal bovine serum and 2 mM L-glutamine. Dependent upon cell doubling time, between 5,000 and 40,000 cells were inoculated into 96 well microtiter plates in a volume of 100 uL per well. The plates were incubated at 37° C., 5% CO₂, 95% air, and 100% relative humidity for 24 hours prior to the addition of the experimental drug. After the 24 hour incubation, two (2) plates of each cell line were fixed in situ with trichloroacetic acid (TCA) to establish the cell population at time of drug addition (Tz).

Prior to use, the experimental drugs were solubilized in dimethyl sulfoxide at 400-fold the desired final maximum test concentration and frozen. At time of drug addition, an aliquot of frozen concentrate was thawed and diluted to twice the desired final maximum test concentration with complete medium containing 50 ug/ml gentamicin. An additional four (4) 10-fold or ½ log serial dilutions was made for a total of five (5) drug concentrations plus a control. An aliquot of 100 ul of each drug dilution was added to the appropriate well that already contains 100 ul of medium containing the cells. The plates were then incubated for 48 hours at 37° C., 5% CO₂, 95% air and 100% relative humidity.

The number of viable cells was estimated by either a SRB or MTT colorimetric assays.

For SRB assay, cells were fixed in situ by the gentle addition of 50 ul cold (w/v) TCA (final concentration, 10% TCA) and incubated for 60 minutes at 4° C. The supernatant was discarded, plates washed five (5) times with tap water and air dried. Then Sulforhodamine B (SRB ) solution (100 μl) at a 0.4% (w.v.) in 1% acetic acid was added to each well, and the plates incubated for 10 minutes at room temperature. Unbound dye was removed by washing five (5) times with 1% acetic acid and the plates were air dried. The bound stain was solubilized with 10 mM trizma baseanad the absorbance was read on an automated plate reader at a wavelength of 515 nm.

The MTT assay measures the ability of viable cells to reduce a tetrazolium salt (MTT) to an insoluble form, a formazan salt. For MTT analysis 20 μl of MTT solution (5 mg/ml in PBS) was added to each well containing cells. The plate was incubated at 37° C. for 5 hours. Media was removed with needle and syringe. 200 μl of DMSO was added to each well and pipetted up and down to dissolve crystals. Plate was placed into a 37° C. incubator for 5 minutes to dissolve air bubbles. Absorbance was read on an automated plate reader at a wavelength of 550 nm.

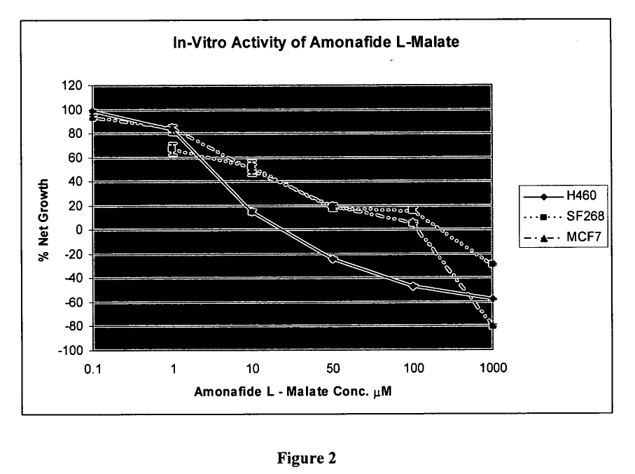

Percent net growth inhibition, as shown in FIGS. 2-7, was calculated using the seven absorbance measurements: absorbance by cells at time zero (Tz), absorbance by cells grown in the absence of drug (C), and absorbance of cells grown at the five drug concentrations (Ti). The following expression was used:

% Net Growth=[(Ti−Tz)/(C-Tz)]×100 if Ti was greater than or equal to Tz.

% Net Growth=[(Ti−Tz)/Tz]×100 if Ti was less than Tz.

Using the plot of Percent Net Growth as a function of drug concentration, lethality concentration “50%” (LC₅₀), total growth inhibition (TGI) and growth inhibition “50%” (GI₅₀) were determined as follows.

TGI was determined as the concentration of Amonafide malate at which Percent Net Growth was 0%. GI₅₀ was determined as the concentration of Amonafide malate at which Percent Net Growth was 50%. LC₅₀ was determined as the concentration of Amonafide malate at which Percent Net Growth was −50%.

As can be seen in FIG. 2, Amonafide malate significantly affected the growth rate of cell lines H-460 (non-small cell lung carcinoma), SF-268 (glioblastoma) and MCF-7 (breast cancer) resulting in GI₅₀ of between 4 and 8 μM. The data presented in Table 5 summarizes the results shown in FIG. 2

TABLE 5

| | H460 | SF268 | MCF7 |
|---|---|---|---|
| GI₅₀ (M) | 4.11E−06 | 7.70E−06 | 5.16E−06 |
| TGI (M) | 0.96E−04 | 1.54E−04 | 1.10E−04 |
| LC₅₀ (M) | 1.70E−04 | 8.42E−04 | 6.62E−04 |

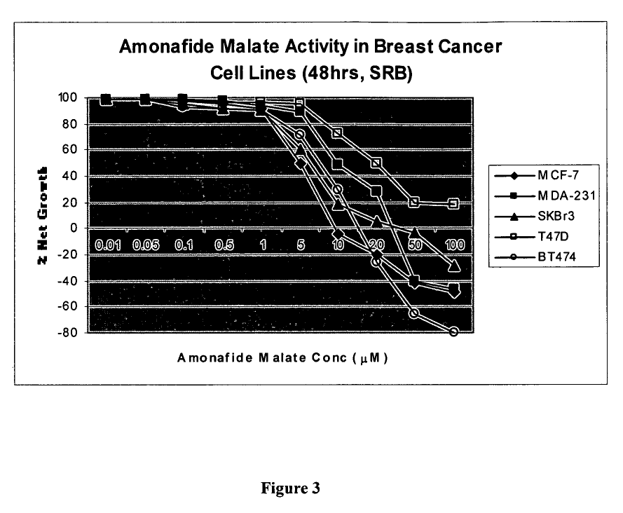

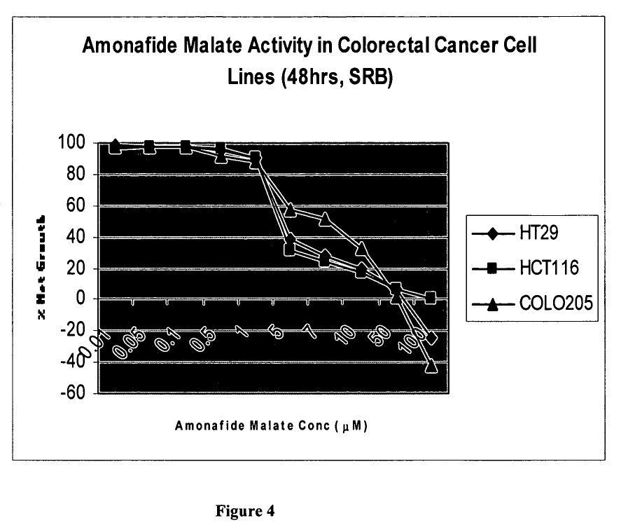

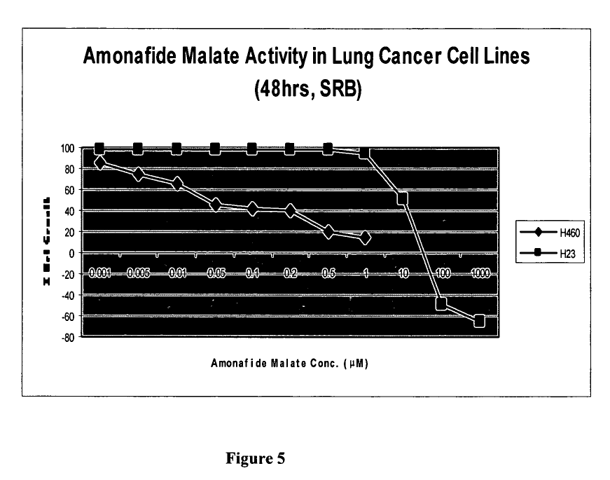

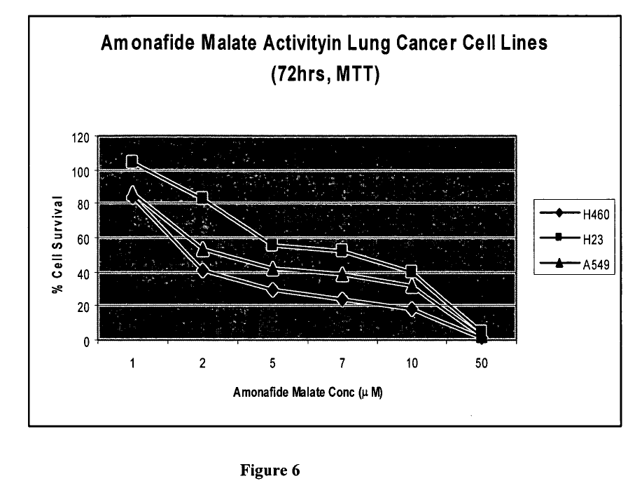

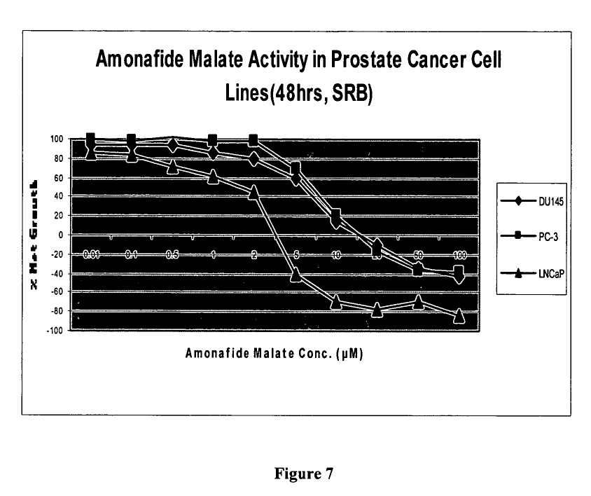

FIGS. 3 to 7 present data for panels of cell lines derived from breast cancer (FIG. 3), colorectal cancer (FIG. 4), lung cancer (FIG. 5, using SRB assay, and FIG. 6, using MTT assay) and prostate cancer (FIG. 7). As can be seen, reduction of net growth by as much as 50% or better was achieved at Amonafide malate concentrations between about 5 and about 10 μM.

### Example 5

### Amanofide has Anti-Cancer Activity In Vivo

Amonafide Malate was tested for in vivo anti-cancer activity in models of three different solid tumor types, MCF7 and MDA-231 (Breast), COLO205 (Colorectal) and PC3 (Prostate). The in vivo activity was tested using the Hollow Fiber methodology developed by the National Cancer Institute: Developmental Therapeutics Branch for use in their antineoplastic agent screening program. Specifically, the anti-cancer activity of amonafide L-malate was tested by the following protocol.

Polyvinylidene fluoride (PVDF) hollow fibers were used. The fibers were individually flushed and filled with 70% ethanol and incubated in 70% ethanol at room temperature for a minimum of 96 hour. Following three washes with deionized water, the fibers were filled with and placed into a pan of deionized water for sterilization by autoclaving. Then the fibers were stored in water at 4° C. until used.

Mice were grouped into 4 groups, two control groups with 6 mice per group and 2 experimental groups with 3 mice per group. The experimental groups were as follows:
Group A: Control, hollow fibers without cells
Group B: Control, hollow fibers for 3 cell lines without drug treatment
Group C: hollow fibers for 3 cell lines+Amonafide Malate

Anesthesia was induced in mice by ketamine/acepromazine/xylazine injected intraperitoneally (i.p.) For the intraperitoneal (i.p.) implants, a small incision was made through the skin and musculature of the dorsal abdominal wall, the fiber samples were inserted into the peritoneal cavity in a craniocaudal direction and the incision was closed with a skin staple. For subcutaneous (s.c.) implants, a small skin incision was made at the nape of the neck to allow insertion of an 11 gauge tumor implant trocar. The trocar, containing the hollow fiber samples, was inserted caudally through the subcutaneous tissues and the fibers were deposited during withdrawal of the trocar. The skin incision was closed with a skin staple. Each mouse was host of 6 samples, representing 3 tumor cells lines each of which was cultured in the 2 physiologic compartments (i.p. and s.c.).

In a first series of experiments, Amonafide Malate (29.4 mg/kg) was injected intraperitoneally (i.p.), once daily on days 3-8. In a second series of experiments, Amonafide Malate was administered i.p. twice-daily at either 15 mg/kg or 29 mg/kg.

On the day following the last drug injection, the animals were sacrificed and the hollow fibers extracted. The hollow fibers were then subjected to the stable endpoint MTT assay as described in Example 4. The optical density of each sample is determined spectrophotometrically at 540 nm and the mean of each treatment group is calculated. The percent net growth for each cell line in each treatment group is calculated as described in Example 4 and compared to the percent net growth in the vehicle treated controls (Group B) using the formula below to determine the percent growth inhibition:
% growth inhibition=(Net Growth_{Control}−Net Growth_{Treated})/Net Growth_{Control})*100

Individual mouse body weights were recorded daily and animals were monitored daily for general health for 6 days. It was not necessary to sacrifice any animals in a CO₂ chamber prematurely as a result of a body weight loss of greater than 20%, or as a result of evidence of other signs of toxicity.

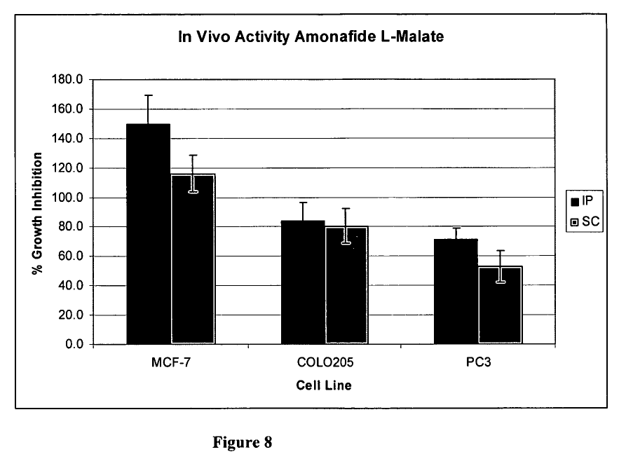

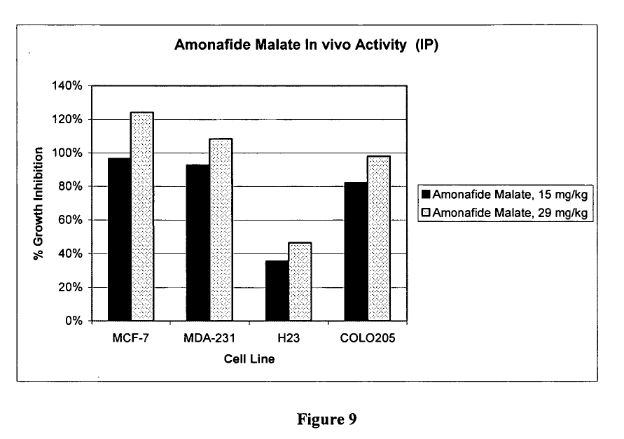

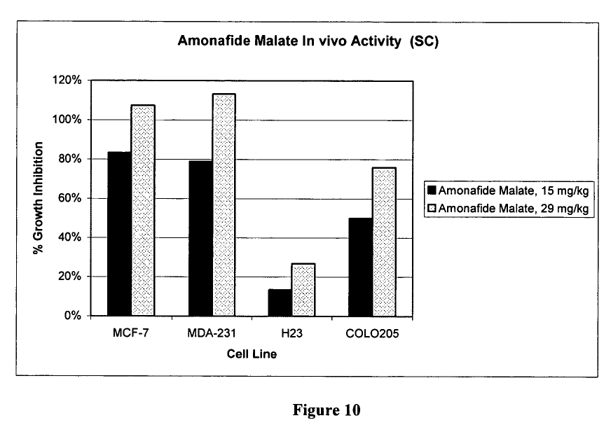

FIG. 8 and Table 6 show data for once-daily administration of 29.4 mg/kg of Amonafide Malate. FIGS. 9 and 10 and Tables 7 and 8 show data for twice-daily administration of Aminofide Malate at either 15 mg/kg or 29 mg/kg.

As presented in Tables 6-8 and FIGS. 2-10, Amonafide malate showed considerable activity, resulting in percent growth inhibition of above 100% (indicating lethality for cancerous cells) for cell lines MCF-7 and MDA-231 (breast cancer). Significant percent growth inhibition was achieved in cell lines PC-3 (prostate) and COLO205 (colorectal cancer).

TABLE 6

| | |
|---|---|
| IP | SC |

| | % Growth | | % Growth | |
|---|---|---|---|---|
| | Inhibition | STDEV | Inhibition | STDEV |
| MCF-7 | 150.2 | 19.2 | 116.1 | 12.1 |
| COLO205 | 84.6 | 11.7 | 80.4 | 11.7 |
| PC3 | 71.6 | 7.3 | 52.9 | 10.9 |

TABLE 7

| | | I.P. fibers | |
|---|---|---|---|
| | | % Growth Inhibition | |
| Cell line | | Amonafide Malate, | Amonafide Malate, |
| Type | Name | 15 mg/kg | 29 mg/kg |
| Breast | MCF-7 | 97% | 124% |
| Breast | MDA-231 | 93% | 108% |
| Lung | H23 | 36% | 47% |
| Colon | COLO205 | 82% | 98% |

TABLE 8

S.C. fibers
% Growth Inhibition

| Cell line | | Amonafide | Amonafide |
|---|---|---|---|
| Type | Name | Malate, 15 mg/kg | Malate, 29 mg/kg |
| Breast | MCF-7 | 83% | 107% |
| Breast | MDA-231 | 79% | 113% |
| Lung | H23 | 13% | 27% |
| Colon | COLO205 | 50% | 76% |

### Example 6

### Organic Hydroxycarboxylic Salts of Amonafide are Produced at Higher Purity than Inorganic Salts Independently from Their Method of Preparation

1. Synthesis of Organic and Mineral Acid Salts of Amonafide by the Method of Example I
   Reaction Yields and Product Purity Directly from the Reaction Pot.

Scheme (I), shown in FIG. 1 shows the sequence of synthetic steps according to the method of Example I. Counterion is exemplified by malate.

The procedure of Example 1 was carried out on a 0.041 mole scale, namely, starting with 10 gram of 3-nitro-1,8-nitronaphthalic anhydride. After formation of the compound (2), while the reaction was still hot, and in triplicate separate experiments, 1.1 molar equivalents of the following organic acids were added: citric, glycolic, lactic, malic, tartaric and the following mineral acids: hydrochloric, and methanesulfonic. The resulting mitonafide salts (compound (2)) were harvested, hydrogenated in 3.5 ml/mmol degassed, deionized water for 12 hours with 5 mg/mmol of 10% Pd/C resulting in compound (3). The reactions were carried out in cohorts of 3. For workup, the resulting suspensions were treated with activated carbon (135 mg/mmol), brought to boil, and filtered free of catalyst while hot. Upon cooling, the mass of precipitated solids were filtered, rinsed with diethyl ether, and vacuum dessicated. Crude yields and purities by HPLC were noted.

The solids obtained as described above were then recrystalized once from the minimum volume of boiling water/ethanol at (1.5/4 vol/vol). The purified salts (compounds (4), Scheme I, FIG. 1) were harvested upon cooling, weighed for yield determination, and analyzed again for purity by HPLC, as previously described. We note that for the purpose of this experiment, the crude products from the above-referenced parallel syntheses were pooled prior to recrystallization. Only the malate and the hydrochloride salts were recrystallized in triplicate.

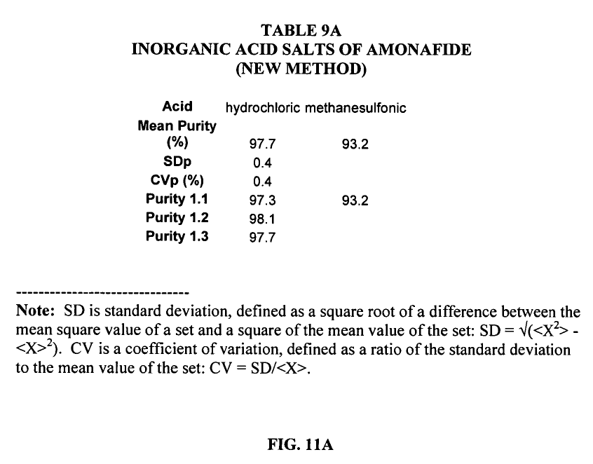

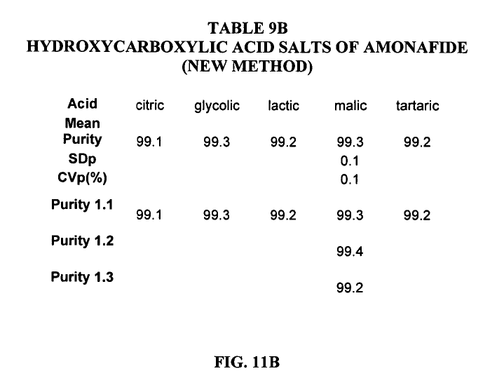

Results of these experiments are presented in FIGS. 11A and 11B, which show Table 9A (inorganic acid salts), and Table 9B (organic hydroxy acid salts), respectively, with mean and standard deviations for purities of malic and hydrochloric acids.

As can be seen from FIGS. 11A and 11B, the hydroxyl acids in each case had the purities higher than the best of the mineral acids (hydrochloric).

Moreover, in the case of the malate and the hydrochloride, the intra-sample means of three replicates afford confirmatory statistics. The mean purity value of the malate salt, 99.3±0.3, exceeds the mean purity of the hydrochloride salt, 97.7±0.4, with a significance of 0.0026 given the one tailed α=0.05.

Furthermore, the mean purity of the hydroxycarboxylic acid cohort is 99.2±0.1, which is better than the mean purity the best of the mineral acids (hydrochloric, 97.7 with a standard deviation of 0.4).
2. Synthesis of Organic and Mineral Acid Salts of Amonafide by the Method of Zee-Cheng et al., U.S. Pat. No. 4,614,820

As used herein, the method of “Zee-Cheng” refers to a method of synthesizing amonafide and salts thereof disclosed in U.S. Pat. No. 4,614,820. The application of this method to the compounds of the instant invention is described in Section V of Example 3 of the instant specification.

Scheme (II), FIG. 1, shows the conceptual sequence of synthetic steps according to the method of Zee-Cheng. Counterion is exemplified by malate.

The procedure of Zee-Cheng as described in Section V of Example 3 of the instant specification was followed on 0.41 reaction scale. Namely, we started with 100 gram of 3-nitro-1,8-nitronaphthalic anhydride. The resulting mitonafide free base (compound (1), scheme II) was taken without further purification from the reaction mixture, as described in the above referenced patent, was subdivided into 5 gram portions. These were then treated in separate experiments with 1.1 molar equivalents of the following organic acids: citric, glycolic, lactic, malic, and tartaric and of the following inorganic acids: hydrochloric, and methanesulfonic. The acids were dissolved in 50 ml of water and hydrogenated with 10% Pd/C for 1 hr at room temperature, resulting in compound (3), scheme II. The resulting solutions were filtered free of catalyst, concentrated to a solid and dissolved in the minimum volume of boiling water/ethanol at (1.5/4 vol/vol). The recrystallized salts were harvested upon cooling, dried under vacuum, weighed to record yield and analyzed again for purity by HPLC, as previously described.

The results are presented in FIGS. 15A and 15B, which show Table 11A (organic salts) and Table 11B (inorganic salts), respectively, with individual run yields, group means and standard deviations.

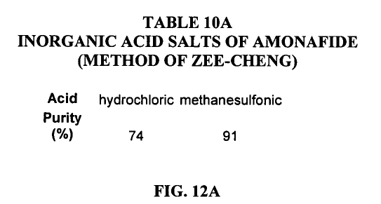

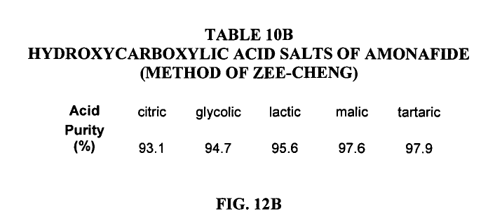

With reference to Tables 10A and 10B (FIGS. 12A and 12B), we note that the amonafide salts of hydroxycarboxylic acids (citric, glycolic, lactic, malic and tartaric acids), even when prepared by the method of Zee-Cheng (Scheme (II), FIG. 1), have purities higher than the purity of the best of the mineral acid (methanesulfonic).

Furthermore, the cohort of the hydroxycarboxylic acids (citric, glycolic, lactic, malic and tartaric acids) showed mean purity (95.8%, standard deviation of 2.0) that are better than the better of the inorganic acid salts (methanesulfonic, 91%).

The results described in Example 6 demonstrate that hydroxycarboxylic acid salts of amonafide, whether prepared by the inventive method of the instant invention or by the method of Zee-Cheng, possess unexpectedly superior purities when contrasted to hydrochloric and methanesuflonic acids. Furthermore, the mean purities of a cohort that comprises citric, glycolic, lactic, malic, and tartaric acid salts, have statistically significantly greater value than purities of either hydrochloric or methansulfonic acid salts.

While this invention has been particularly shown and described with references to preferred embodiments thereof, it will be understood by those skilled in the art that various changes in form and details may be made therein without departing from the scope of the invention encompassed by the appended claims.

1. An organic acid salt of amonafide wherein the organic acid is selected from the group consisting of a hydroxy C2-C6 aliphatic monocarboxylic acid, a hydroxy C3-C8 aliphatic dicarboxylic acid and a hydroxy C4-C10 tricarboxylic acid.

2. The organic acid salt of claim 1 wherein the organic acid is citric acid, glycolic acid, lactic acid, malic acid, or tartaric acid.

3. The organic acid of claim 1 wherein amonafide is in the monovalent form.

4. A pharmaceutical composition comprising a pharmaceutically acceptable carrier or diluent and an organic acid salt of amonafide, wherein the organic acid is selected from the group consisting of a hydroxy C2-C6 aliphatic monocarboxylic acid, a hydroxy C3-C8 aliphatic dicarboxylic acid and a hydroxy C4-C10 tricarboxylic acid.

5. The pharmaceutical composition of claim 4 wherein the organic acid is citric acid, glycolic acid, lactic acid, malic acid, or tartaric acid.

6. The pharmaceutical composition of claim 4 wherein amonafide is in the monovalent form.

7. A method of treating a subject with cancer selected from the group consisting of breast cancer, colon cancer, lung cancer, prostate cancer and leukemia, comprising the step of administering to the subject an effective amount of an organic acid salt of amonafide, wherein the organic acid is a C2-C6 aliphatic hydroxyl monocarboxylic acid, a C3-C8 aliphatic hydroxyl dicarboxylic acid or a C4-C10 aliphatic hydroxyl tricarboxylic acid.

8. The method of claim 7 wherein the organic acid is citric acid, glycolic acid, lactic acid, malic acid, or tartaric acid.

9. The method of claim 7 wherein amonafide is in the monovalent form.
